(19) United States
(12) Patent Application Publication
(10) Pub. No.: US 2022/0257356 A1
(43) Pub. Date: Aug. 18, 2022

(54) Evacuation Apparatus and Method

(71) Applicant: M.N. Parchewsky Professional Corporation, Calgary (CA)

(72) Inventors: Julie PARCHEWSKY, Calgary (CA); Michael PARCHEWSKY, Calgary (CA)

(21) Appl. No.: 17/671,427

(22) Filed: Feb. 14, 2022

Related U.S. Application Data
(60) Provisional application No. 63/179,624, filed on Apr. 26, 2021.
(60) Provisional application No. 63/148,738, filed on Feb. 12, 2021.

(51) Int. Cl. A61C 17/06; A61C 17/08

(52) U.S. Cl. CPC A61C 17/096; A61C 17/08; A61C 17/065

(57) ABSTRACT

A suction tool is provided for evacuating debris, liquids, aerosols, and the like. The suction tool has a central lumen or bore, and one or more peripheral flow passages that are in communication with the central lumen. A low volume evacuation tool, such as a saliva ejector, can be connected or integrated with the central lumen for evacuating saliva and debris. The peripheral flow passages evacuate aerosols and other airborne particles. The suction tool can be connected with conventional vacuum pumps. The suction tool can have a control port that can be opened or obstructed to control the flow rate through the central lumen and the flow passages.

### CROSS-REFERENCE TO RELATED APPLICATIONS

This application claims the benefit of U.S. Provisional Application 63/179,624 filed Apr. 26, 2021; and Provisional Application 63/148,738 filed Feb. 12, 2021, the entirety of each of which are incorporated fully herein by reference.

### FIELD

Embodiments herein relate to apparatus and methods for dental procedures. More particularly, embodiments herein relate to an improved apparatus for evacuating fluids, debris, and aerosols during dental procedures and methods for using same.

### BACKGROUND

Suction tools are commonly used to remove debris, saliva, and aerosols during dental and dental hygiene procedures (herein referred to generally as “dental procedures”). Such suction tools comprise a hand tool connected to a suction source such as a vacuum pump. Suction tools can generally be categorized as low-volume evacuation (LVE) tools and higher flow high-volume evacuation (HVE) tools. While effective at removing fluids and debris, LVE tools such as saliva ejectors are largely ineffective at removing aerosols and other particulates generated by certain procedures. Additionally, LVE tools present a risk of backflow into the patient's mouth, for example in situations where pressure in the patient's mouth is lower than that of the suction source, such as when the patient closes their mouth around the tip of the tool.

Procedures in which powered instruments, such as ultrasonic scalers and air polishers, are used are known to generate aerosols that can spread throughout the operatory and land on various surfaces, or even further such as into the ventilation system. The spread of such aerosols is undesirable, as pathogens such as bacteria and viruses can be carried therein. Thus, aerosol-generating procedures can be detrimental to sanitation and can threaten the health of patients and clinicians alike if the aerosols are not removed effectively.

There has been an increasing need for high volume evacuation (HVE) suction tools capable of removing aerosols generated during dental procedures. HVE suction tools are also connected to a suction source and are capable of evacuating air and fluids at a higher rate relative to LVE tools, for example at 100 CFM. HVE tools require significantly more vacuum pressure than LVE tools, for example requiring up to six times greater vacuum pressure. HVE tools are typically held near the work area to remove aerosols produced during dental procedures. While effective at removing aerosols, HVE tools are loud and can be uncomfortable for the patient. For example, the HVE tool can “catch” if it contacts the intraoral mucosa or other tissue of the patient, causing discomfort.

LVEs are still preferred over HVEs in certain contexts, as they permit the convenient removal of residual fluid and debris accumulated during dental procedures easily and comfortably by simply having the patient close their mouth.

Both HVE and LVE tools can be used in four-handed dentistry procedures, for example wherein a dental assistant can use both HVE and LVE suction tools while the dentist or dental hygienist performs the procedure. Such an arrangement provides the advantages of both HVE and LVE.

However, in two-handed procedures where only one dental practitioner is present, it is impractical for the practitioner to operate multiple suction devices while also performing the procedure. Such is often the case during dental hygiene procedures, where a dental hygienist is independently performing the procedure on the patient. During dental hygiene procedures, hygienists will often use ultrasonic scaling devices that produce aerosols. As such, an HVE suction device is desirable to remove aerosols. At the same time, an LVE tool is also desirable to control saliva build-up in a convenient manner without causing discomfort to the patient. While it is possible for the hygienist to use an HVE tool with one hand and the scaling device in the other, the HVE device must be used to evacuate saliva in addition to aerosols. The proximity of the HVE device to the patient's cheek, tongue, or other tissue can cause the device to catch and cause discomfort. The need to be mindful of the placement of the HVE also makes the procedure cumbersome. Such drawbacks may be addressed by having a second practitioner, such as a dental assistant, operate the HVE during the procedure while the hygienist operates the LVE, or vice versa. However, such an arrangement is uneconomical for most dental practices.

There is interest in the industry for an improved evacuation apparatus that effectively removes aerosols, fluids, and debris during dental procedures without discomfort to the patient, and without the need for additional personnel.

### SUMMARY

Embodiments of a suction device for use in dental procedures are disclosed herein. The suction device is configured to be connected to a suction source, e.g. a vacuum pump, at a first connection. In embodiments, the suction device has a central lumen extending therethrough and in communication with the first connection, and one or more peripheral passages arranged around and generally parallel with the central lumen and also in communication with the first connection. In embodiments, the at least one peripheral passage is in communication with the central lumen, such as toward the first connection. In embodiments, the suction device is further configured to couple with a low-volume evacuation (LVE) tool, such as a saliva ejector. The LVE tool can be received in the central lumen, such that the suction device provides a means for LVE via the central lumen and also HVE capability via the one or more peripheral passages.

In a first aspect, a suction device is provided for connection to a suction source, comprising: a body having a first end and a second end opposite the first end; a first connection located at the first end configured to couple with the suction source; a central lumen in communication with the first and second end and a common bore toward the first end; and one or more peripheral flow passages each terminating at a respective peripheral opening toward the second end and in communication with the common bore toward the first end.

In an embodiment, the central lumen is configured to receive a low volume evacuation (LVE) tool at about the second end.

In an embodiment, the LVE tool extends beyond the second end of the suction device by a length.

In an embodiment, the length by which the LVE tool extends beyond the second end of the suction device is adjustable.

In an embodiment, the body further comprises a control port for selectably permitting air flow between the environment and the common bore, wherein obstructing the control port increases air flow through the central lumen and peripheral flow passages.

In an embodiment, the control port is sized such that it may be obstructed by a finger.

In an embodiment, the suction device further comprises a control valve configured to control air flow through the control port, the control valve capable of actuating between at least a fully open position and a fully closed position.

In an embodiment, the control valve is capable of actuating to one or more intermediate positions.

In an embodiment, the body comprises an inner tubular portion residing in an outer tubular portion, the central lumen extending through the inner tubular portion and the one or more peripheral flow passages defined between the inner tubular portion and the outer tubular portion.

In an embodiment, the body comprises a lumen wall having the central lumen extending therethrough, and the one or more peripheral flow passages are formed on the outside of the lumen wall and are in communication with the common bore through one or more peripheral ports of the lumen wall.

In an embodiment, the second end of the device is beveled.

In an embodiment, the device is made of silicone.

In an embodiment, the device is made of an autoclavable material.

In an embodiment, the suction source is capable of removing 100CFM of air.

In another broad aspect, a suction device is provided for connection to a suction source, comprising: a first end and a second end opposite the first end; an inner tubular portion supported in an outer tubular portion with one or more support structures; a central lumen extending through the inner tubular portion; one or more peripheral passages defined between the inner tubular portion and the outer tubular portion, the one or more peripheral passages in communication with the central lumen toward the first end; wherein the central lumen and one or more peripheral passages are in communication with a common bore toward the first end.

In an embodiment, the central lumen is configured to receive a low volume evacuation (LVE) tool at about the second end.

In an embodiment, the suction device further comprises a control port formed in the outer tubular portion for selectably permitting air flow between the environment and the common bore, wherein obstructing the control port increases air flow through the central lumen and peripheral flow passages.

In an embodiment, the suction device further comprises a control valve configured to control air flow through the control port, the control valve capable of actuating between at least a fully open position and a fully closed position.

In an embodiment, the one or more support structures comprise two longitudinal seams to define two peripheral flow passages.

In another broad aspect, a method of evacuating fluid, debris, and aerosols comprises: connecting a suction tool to a suction source such that air flows through a central lumen and one or more peripheral flow passages of the suction tool toward the suction source; removing fluids and debris through the central lumen; and removing aerosols through the one or more peripheral flow passages.

In an embodiment, the method further comprises selectably obstructing a control port of the suction tool to modulate the rate of air flow through the central lumen and the one or more peripheral flow passages.

### BRIEF DESCRIPTION OF THE DRAWINGS

FIG. 1A is a perspective view of an embodiment of a suction device described herein;

FIG. 1B is a side elevation view of the suction device of FIG. 1A;

FIG. 1C is a side elevation view of the suction device of FIG. 1B rotated 90 degrees;

FIG. 1D is a side elevation view of the suction device of FIG. 1B rotated 180 degrees;

FIG. 2A is a side elevation view of the suction device of FIG. 1A having an LVE tool inserted into a central lumen thereof;

FIG. 2B is a perspective view of the suction device of FIG. 2A;

FIG. 2C is a side elevation view of the suction device of FIG. 2A rotated 90 degrees;

FIG. 2D is a side elevation view of the suction device of FIG. 2A rotated 180 degrees;

FIG. 3 is a close-up perspective view of the suction device of FIG. 2A;

FIG. 4A is a side elevation partial cross-sectional view of a suction device coupled with a suction line and a saliva ejector;

FIG. 4B is a side elevation partial cross-sectional view of a suction device coupled with a saliva ejector;

FIG. 5 is a fanciful partial cross-sectional top plan view of a suction device coupled with an LVE tool showing the flow paths of the HVE and LVE portions of the suction tool;

FIG. 6A is a front view of an embodiment of a suction device having an inner tubular portion secured to an outer tubular portion at two seams to define peripheral flow passages;

FIG. 6B is a front view of an embodiment of a suction device having an inner tubular portion secured to an outer tubular portion at one seam to define a peripheral flow passage; and

FIG. 6C is a front view of an embodiment of a suction device having four peripheral flow passages positioned about a central lumen wall.

### DETAILED DESCRIPTION

Embodiments are described herein in the context of dental procedures. However, as one of skill in the art would understand, the systems, apparatus, and methods disclosed herein are also applicable in other procedures wherein it is desired to remove fluids, debris, and aerosols.

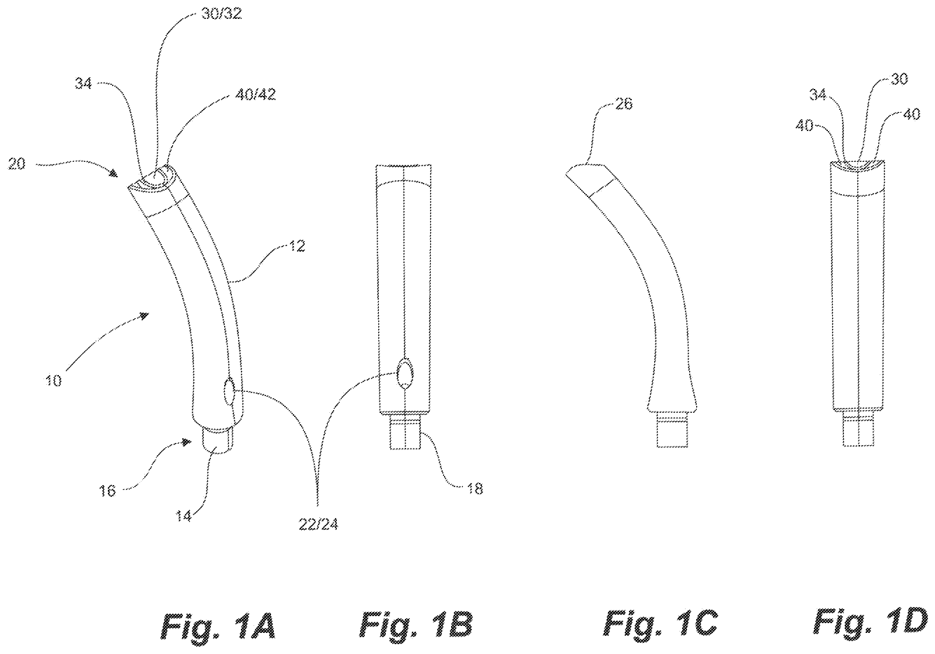

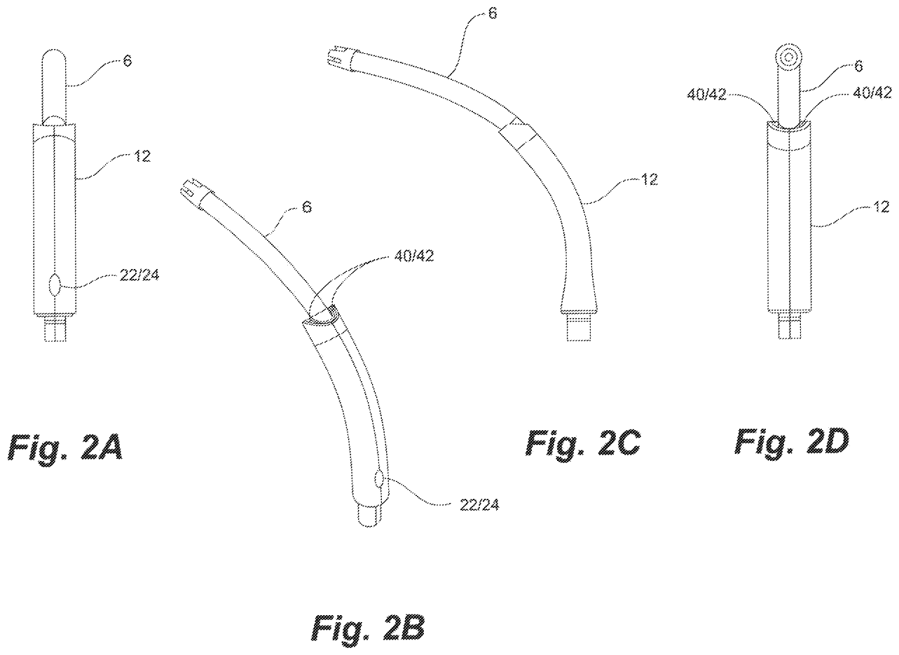

With reference to FIGS. 1A-2D, in an embodiment, an improved suction device 10 comprises a body 12 having a first connection 14 located at a first or suction end 16 thereof, the first connection 14 configured to be coupled to a suction line 8, which is in turn connected to a vacuum or suction source (not shown). A second or patient end 20 is located opposite the first end 16 of the body. The body 12 further comprises a central bore or lumen 30 extending therethrough and in communication with the first and second ends of the body 16, 18. At the second end 20 of the body, the central lumen 30 terminates at central opening 32. Toward the first end 16 of the body, the central lumen 30 is in communication with, or is co-extensive with, a common bore 38 which is in communication with the first connection 14. In embodiments, the first connection 14 can be sized to accommodate standard suction hose sizes, such as ¼″ or ⅝″ HVE hoses. In the depicted embodiment, the body 12 is curved to provide improved ergonomic access to the patient's oral cavity. In other embodiments, the body 12 can be straight, angled, or be provided in any other desired shape without departing from the scope of the present invention.

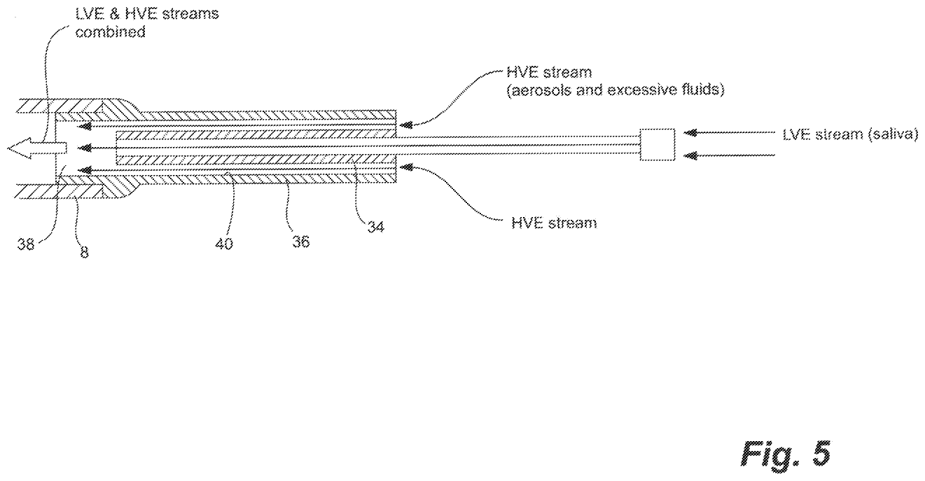

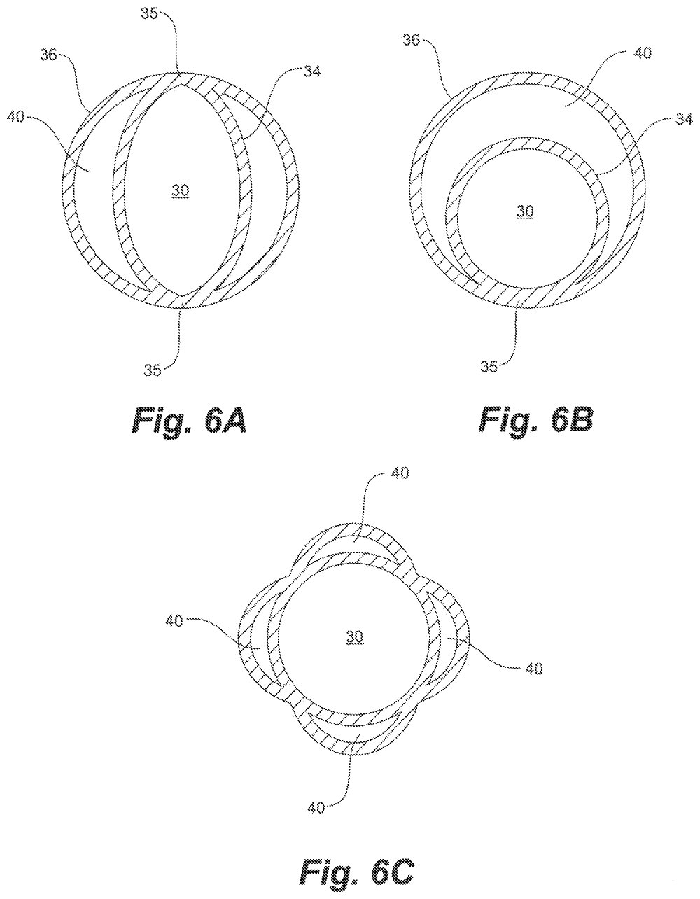

The body 12 further comprises one or more peripheral flow passages 40 running generally parallel to the central lumen 30. A portion of each of the peripheral flow passages 40 is separated from the central lumen 30 by a lumen wall 34. As best shown in FIG. 5, the peripheral flow passage 40 joins with the central lumen 30 toward the first end 16 of the suction device 10 at common bore 38, such that the one or more peripheral flow passages 40 and central lumen 30 are in communication or are co-extensive with each other toward the first end 16. Each peripheral flow passage 40 terminates at a respective peripheral opening 42 toward the second end 20 of the body 12. As shown in FIGS. 1A-2D, 4A, 4B, and 5, in an embodiment, two peripheral flow passages 40 run parallel with central lumen 30 and join with the central lumen 30 toward the first end 16 of the suction device 10, forming common bore 38. In other embodiments, as shown in FIG. 6C, the one or more peripheral flow passages 40 are arranged around the central lumen 30 and are in communication with the central lumen 30 via peripheral ports formed in the lumen wall 34 toward the first end 16.

The one or more peripheral flow passages 40 can be formed in multiple ways. For example, as shown in FIGS. 1A-2D and 6A, the peripheral flow passages 40 are located on opposing sides of the central lumen wall 34 and joined lengthwise therewith along first and second seams 35. In other words, the lumen wall 34 forms an inner tubular portion residing in an outer tubular portion 36, and the inner tubular portion/lumen wall 34 is supported in the outer tubular portion 36 along first and second seams 35, thus defining the two peripheral flow passages 40 located along opposing sides of the lumen wall 34. The seams 35 joining the lumen wall 34 and outer tubular portion 36 can be continuous, or can have one or more openings such that the peripheral flow passages 40 are in communication with each other. In some embodiments, other support structures besides seams 35 can be used to join the lumen wall 34 and outer tubular portion 36, such as weld beads, radially extending supports, and the like.

In other embodiments, with reference to FIG. 6B, the inner tubular/lumen wall 34 can be joined with the outer tubular portion 36 along one side only, such that a peripheral flow passage 40 is formed around the central lumen 30 and has a crescent-shaped cross-sectional flow area.

In still other embodiments, with reference to FIG. 6C, the multiple peripheral passages 40 are arranged around the lumen wall 34.

In all the above embodiments, the one or more peripheral flow passages 40 are in communication with the common bore 38 toward the first end 16 of the suction tool 10, such that the tool 10 can be connected with a single suction source at the first connection 14 for drawing air through both the central lumen 30 and peripheral flow passages 40. Other suitable arrangements of the peripheral flow passages 40 are possible without departing from the scope of the present invention.

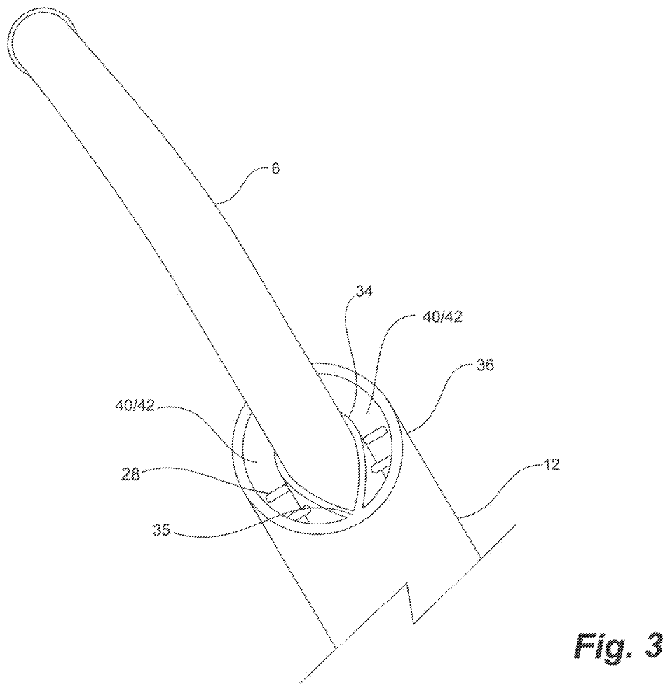

As shown in FIG. 3, in some embodiments, one or more bracing structures 28 can be provided in the peripheral flow passages 40 to brace the walls of said flow passages 40 against the lumen wall 34 and prevent the peripheral flow passage 40 from collapsing, for example due to suction or the force of the hand of a user gripping the suction tool 10.

In embodiments, the cumulative cross-sectional flow area of the peripheral flow passages 40 can be configured to be greater than the cross-sectional flow area of the central lumen 30, such that air flows through the peripheral flow passages 40 at a higher rate than through the central lumen 30. In such a configuration, the peripheral flow passages 40 function as the HVE portion of the suction tool 10 while the central lumen 30 functions as the LVE portion.

In embodiments, with reference to FIGS. 1A-2D, 4A, and 4B, a control port 22 can be formed in the body 12 and enable communication between the common bore 50 and the exterior of the body 12 via the control port 22. As depicted, the control port 22 is an aperture in communication with the common bore 38. When unobstructed, the control port 22 provides another flow path for air to be drawn into the common bore 38, thereby decreasing the flow through the central and peripheral openings 32,42. When the control port 22 is partially or fully obstructed, for example by the finger of an operator, air can only be drawn through the central and peripheral openings 32,42, thereby increasing the flow through the central lumen 30 and peripheral flow passages 40. In some embodiments, a control valve 24 can be provided to control air flow through the control port 22. For example, a ball valve, butterfly valve, gate valve, or any other suitable valve can be used as a control valve 24. Control valve 24 can be configured to be actuable between a fully open and a fully closed position, and can also be configured to be actuable to one or more intermediate positions to provide finer adjustment of air flow through the control port 22, and in turn modulate air flow through the central and peripheral openings 32,42. In other embodiments, the control port 22 can be in communication with only the peripheral flow passages 40, or both the central lumen 30 and peripheral flow passages 40, as opposed to the common bore 38.

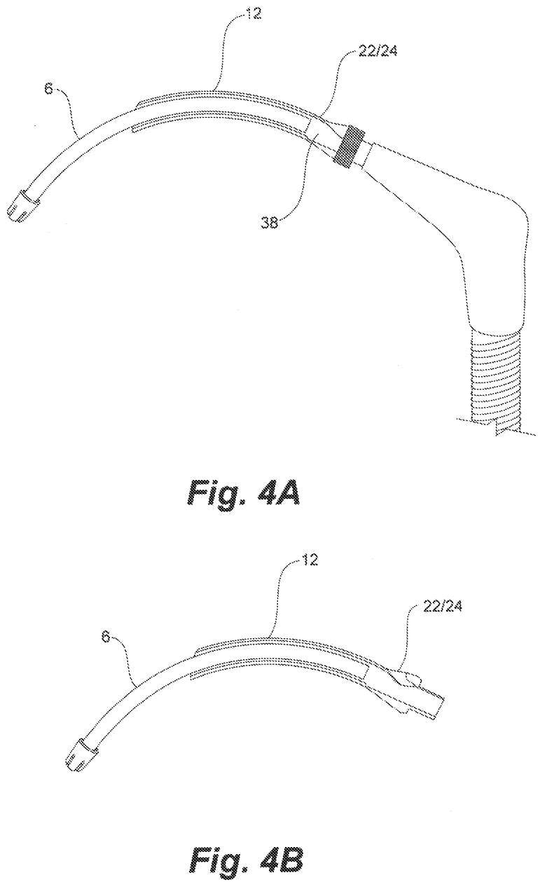

A low volume evacuation tool 6, such as a saliva ejector, can be connected to the central opening 32 of the suction device 10. For example, as shown in FIGS. 4A-4B, a saliva ejector 6 is inserted through the central opening 32 into the central lumen 30 to permit saliva and debris to be drawn from the oral cavity of the patient into the central lumen 30 and out through the suction hose 8.

In use, the central lumen 30 of the suction device 12 can be coupled with an LVE tool 6 such as a saliva ejector while the one or more peripheral openings 42 are left open 40. The first connection 14 of the suction device 10 can be connected to suction tubing or a suction hose 8, which is in turn connected to a suction source such as a suction/vacuum pump (not shown). In an exemplary embodiment, the suction source is a high-volume suction source capable of servicing both LVE and HVE tools. During operation, air is drawn into the common bore 38 through both the central lumen 30 and peripheral flow passages 40. The saliva ejector 6 coupled to the central opening 32 can be used to remove fluid and debris. At the same time, the at least one peripheral opening 42 functions as a HVE suction tool to remove aerosols and excess fluids. In this manner, the present suction tool 10 provides both HVE and LVE functions in a single tool, and can be operated with one hand. Such one-hand operation enables the practitioner to use other dental tools such as ultrasonic scalers and air polishers with their other hand, dispensing with the need for additional personnel to operate multiple suction tools.

Should the need arise for increased suction, for example when power tools such as ultrasonic scalers or air polishers are used, generating increased aerosols, the practitioner can partially or fully close the control port 22 to increase the amount of air drawn through the peripheral openings 42. Suction through the central lumen 30/saliva ejector 6 is also increased by closing the control port 22, which can be utilized to reduce the risk of backflow. In this manner, the suction device 10 mitigates the risk of backflow in two ways: first, by permitting air to continue to flow through the suction device 10 via the peripheral passages 40 when the LVE 6 is closed, for example when the patient's mouth is closed, thereby allowing continued positive airflow through the device, and secondly by providing an HVE component in the form of the peripheral passages 40 to increase the air flow of the overall device 10, which makes it more difficult for the patient to overcome the suction force when they close their mouth over the LVE tool 6. Thus, the patient cannot create a true vacuum force, which would contribute to backflow, as the peripheral passages 40 continue to move fluid and air away from the oral cavity.

In embodiments, the suction device 10 can be provided as a discrete component for coupling with a suction source and LVE tool 6. For example, the diameter of the central lumen 30 can be sized to accommodate a ¼″ saliva ejector 6. In other embodiments, the LVE tool 6 can be formed integrally with the suction device 10. For example, the central lumen 30 can form part of the tubing of a saliva ejector 6, such that the two are integral and coextensive. Moreover, in alternative embodiments, other tools can be connected to the peripheral openings 42/peripheral flow passages 40.

In embodiments, the peripheral openings 40 can be set back a distance from the central opening 32 or tip of the LVE tool/saliva ejector 6. For example, the peripheral openings 42 can be configured to be located near the interface between the patient's oral cavity and the external environment during use to more effectively remove aerosols generated in the oral cavity before they can exit into the environment. In two-handed dental procedures, the peripheral openings 42 can be configured to be positioned at the corner of the patient's mouth, where the aerosols exit the oral cavity, thus removing aerosols and preventing them from exiting into the environment. Applicant has found that, for the removal of aerosols, the ideal distance from an ultrasonic scaler to HVE suction is between 6-15 mm. Thus, the length of the LVE tool 6 and position of the peripheral openings 42 can be selected to maximize the efficiency of aerosol removal during scaling procedures, for example by locating the peripheral openings 6-15 mm away from the tip of the LVE tool. In some embodiments, the length of the LVE tool 6 is adjustable such that the distance between the tip of the LVE tool 6 and the peripheral openings 42 can be varied as needed to properly position the peripheral openings 42 to effectively remove aerosols. For example, with reference to FIGS. 1A-2D and 5, the extent the LVE tool 6 is inserted into the central lumen 30 can be adjusted to vary the distance between the tip of the LVE tool 6 and peripheral openings 42.

In embodiments, the central lumen 30 can also be left open, i.e. with no LVE tool 6 connected to the central lumen 30, such that the suction device 10 provides greater aerosol removal and HVE performance.

In some embodiments, the second end 20 of the suction device 10 can be angled or beveled to reduce tissue irritation and permit better directional access to oral debris and saliva. For example, as shown in FIGS. 1C and 2C, by forming a 45-degree bevel 26 across the second end 20 of the suction device 10, the tissue of the patient can be retracted easier without catching on the suction device. Further, upon retraction, the plane of the second end 20 of the suction device 10 will be about perpendicular to the facial surfaces of the teeth and the aerosol debris field.

In embodiments, the suction device 10 can be made of a disposal material such as plastic, silicone, or other suitable material, such that it can be replaced after every use. In some embodiments, the suction device can be made of a more durable material such as stainless steel, autoclavable plastics such as polypropelyne (PP) and polypropylene copolymer (PPCO), fluoropolymer products such as polytetrafluoroethylene (PTFE), perfluoroalkoxy (PFA), fluorinated ethylene propylene (FEP), or ethylene tetrafluoroethylene (ETFE) such that the device 10 may be cleaned, such as via autoclave, and reused.

A lightweight hose attachment can also be provided for use with the suction device to decrease the strain of using the tool and improve the ease of use of the device. The flexible tubing permits greater weight management and ease-of-use during two-handed procedures, while providing the same suction efficiency.

In embodiments, the angle and length of components of the suction device 10, such as the LVE tool 6, can be adjustable to provide additional versatility.

Standard HVE suction tools typically have a ½″ diameter inlet, with an internal bore diameter of 5/16″. At 5″ Hg of suction pressure, the maximum amount of air that may flow through such tools is 11.07 CFM. At 10″ Hg of suction pressure, the maximum amount of air flow therethrough is 14.64 CFM. Standard LVE suction tools typically have a ¼″ diameter inlet, with an internal bore diameter of ⅛″. At 5″ Hg of suction pressure, the maximum amount of air that may flow through such LVE tools is 2.04 CFM. At 10″ Hg of suction pressure, the maximum amount of air flow therethrough is 2.55 CFM. While the suction device 10 can be configured to accommodate such LVE tools and provide a total flow diameter similar to those of standard HVE tools, the suction device can also be configured to accommodate any size of LVE/HVE tools and provide any desired HVE flow diameter.

The suction device described herein is advantageous as both HVE and LVE functionality is provided in a single tool that may be operated with one hand. Further, when an LVE tool is connected to the central lumen of the suction device, the peripheral openings can be set back from the tip of the LVE tool to avoid contact thereof with the cheek or tongue of the patient, thereby mitigating the likelihood that the peripheral openings will catch on the tissue of the patient. Additionally, the peripheral openings can be positioned an ideal distance from the tip of the LVE tool to provide optimal aerosol removal. Further, the presence of the control port gives the operator control over the level of suction and air flow through the LVE tool and peripheral flow passages, which further mitigates the likelihood of the suction device catching on the tissue of the patient while still providing high flow capability when desired. The ability to increase suction as desired also reduces the potential for backflow.

What is claimed is:

1. A suction device for connection to a suction source, comprising:
a body having
a first end and a second end opposite the first end;
a first connection located at the first end configured to couple with the suction source;
a central lumen in communication with the first and second end and a common bore toward the first end; and
one or more peripheral flow passages each terminating at a respective peripheral opening toward the second end and in communication with the common bore toward the first end.

2. The suction device of claim 1, wherein the central lumen is configured to receive a low volume evacuation (LVE) tool at about the second end.

3. The suction device of claim 2, wherein the LVE tool extends beyond the second end of the suction device by a length.

4. The suction device of claim 3, wherein the length by which the LVE tool extends beyond the second end of the suction device is adjustable.

5. The suction device of claim 1, wherein the body further comprises a control port for selectably permitting air flow between the environment and the common bore, wherein obstructing the control port increases air flow through the central lumen and peripheral flow passages.

6. The suction device of claim 5, wherein the control port is sized such that it may be obstructed by a finger.

7. The suction device of claim 6, further comprising a control valve configured to control air flow through the control port, the control valve capable of actuating between at least a fully open position and a fully closed position.

8. The suction device of claim 7, wherein the control valve is capable of actuating to one or more intermediate positions.

9. The suction device of claim 1, wherein the body comprises an inner tubular portion residing in an outer tubular portion, the central lumen extending through the inner tubular portion and the one or more peripheral flow passages defined between the inner tubular portion and the outer tubular portion.

10. The suction device of claim 1, wherein the body comprises a lumen wall having the central lumen extending therethrough, and the one or more peripheral flow passages are formed on the outside of the lumen wall and are in communication with the common bore through one or more peripheral ports of the lumen wall.

11. The suction device of claim 1, wherein the second end of the device is beveled.

12. The suction device of claim 1, wherein the device is made of silicone.

13. The suction device of claim 1, wherein the device is made of an autoclavable material.

14. A suction device for connection to a suction source, comprising:
a first end and a second end opposite the first end;
an inner tubular portion supported in an outer tubular portion with one or more support structures;
a central lumen extending through the inner tubular portion;
one or more peripheral passages defined between the inner tubular portion and the outer tubular portion, the one or more peripheral passages in communication with the central lumen toward the first end;
wherein the central lumen and one or more peripheral passages are in communication with a common bore toward the first end.

15. The suction device of claim 14, wherein the central lumen is configured to receive a low volume evacuation (LVE) tool at about the second end.

16. The suction device of claim 14, further comprising a control port formed in the outer tubular portion for selectably permitting air flow between the environment and the common bore, wherein obstructing the control port increases air flow through the central lumen and peripheral flow passages.

17. The suction device of claim 16, further comprising a control valve configured to control air flow through the control port, the control valve capable of actuating between at least a fully open position and a fully closed position.

18. The suction device of claim 14, wherein the one or more support structures comprise two longitudinal seams to define two peripheral flow passages.

19. A method of evacuating fluid, debris, and aerosols, comprising:
connecting a suction tool to a suction source such that air flows through a central lumen and one or more peripheral flow passages of the suction tool toward the suction source;
removing fluids and debris through the central lumen; and
removing aerosols through the one or more peripheral flow passages.

20. The method of claim 19, further comprising selectably obstructing a control port of the suction tool to modulate the rate of air flow through the central lumen and the one or more peripheral flow passages.
